(19) United States
(12) Patent Application Publication
(10) Pub. No.: US 2026/0159796 A1
(43) Pub. Date: Jun. 11, 2026

(54) INCUBATOR MICROPLATE RACK

(71) Applicant: ARIZONA BOARD OF REGENTS ON BEHALF OF THE UNIVERSITY OF ARIZONA, Tucson, AZ (US)

(72) Inventor: Matthew E. Kaplan, Tucson, AZ (US)

(21) Appl. No.: 19/414,872

(22) Filed: Dec. 10, 2025

Related U.S. Application Data
(60) Provisional application No. 63/730,255, filed on Dec. 10, 2024.

(51) Int. Cl. C12M 3/00; C12M 1/22

(52) U.S. Cl. CPC C12M 23/48; C12M 23/10; C12M 23/42

(57) ABSTRACT

An incubator rack for cell culture in microplates. The rack system allows for a sturdy system with proper airflow and low thermal conductivity. Data has been generated showing that cells grown in plates incubated in these racks develop more consistently than plates stacked directly.

### CROSS-REFERENCE TO RELATED APPLICATIONS

This application is a non-provisional and claims benefit of U.S. Provisional Application No. 63/730,255 filed Dec. 10, 2024, the specification of which is incorporated herein in its entirety by reference.

### FIELD OF THE INVENTION

The present invention relates to analytical and testing instruments used in cell cultures, namely, an incubation rack for storing microplates of cell cultures.

### BACKGROUND OF THE INVENTION

Reducing plate-to-plate variation when performing cell culture in microplates is an essential goal in cell-based research. Existing incubator racks have solid (often thermally conductive metal) shelves and walls as shown in FIG. 3. This limits air flow around the microplates, as well as increases thermal conductivity often causing the plates closer to the bottom stack to change temperature faster. Other incubator racks only allow for airflow on one or several sides of the microplates while implementing solid surfaces on other sides. Hence, there is a need for incubator racks that can better support consistent growth of cell cultures across multiple microplates.

### BRIEF SUMMARY OF THE INVENTION

It is an objective of the present invention to provide devices that allow for more consistent cell culturing, as specified in the independent claims. Embodiments of the invention are given in the dependent claims. Embodiments of the present invention can be freely combined with each other if they are not mutually exclusive.

In some aspects, the present invention features an incubator rack for providing uniform growth conditions to microplates stored therein. The incubator rack may comprise a plurality of shelves for holding said microplates. Without wishing to limit the invention, the incubator rack can have sufficient shelving space for airflow between and around the microplates.

In other embodiments, the present invention features an incubator rack comprising a first sidewall, a second sidewall parallel to the first sidewall, a bottom shelf connecting the first and second sidewalls, a top shelf connecting the first and second sidewalls, a plurality of rails disposed on an interior surface of each sidewall, a sidewall frame perpendicular to the two parallel sidewalls, the top shelf, and the bottom shelf, and a plurality of holes disposed through the first and second sidewalls.

Without wishing to limit the invention to any theory or mechanism, it is believed that the present invention provides a sturdy rack with even spacing between plates and ample distance from the base to provide unrestricted airflow around the microplates and low thermal conductivity from the floor or shelf of the incubator. Data has been generated showing that cells grown in plates incubated in these racks develop more consistently than plates stacked directly in the incubator (as is common laboratory practice).

One of the unique and inventive technical features of the present invention is the implementation of shelves with exposure holes on all sides (front, back, sides, top, and bottom) such that the microplates on the shelves are fully exposed to airflow around and between them. Without wishing to limit the invention to any theory or mechanism, it is believed that the technical feature of the present invention advantageously provides for uniform growth conditions for microplates, resulting in consistent development. None of the presently known prior references or works have the unique inventive technical feature of the present invention.

Furthermore, the inventive technical feature of the present invention is counterintuitive. The reason that it is counterintuitive is because it contributed to a surprising result. One of ordinary skill in the art would expect that enclosing the microplates on more sides would create a more stable environment and more uniform growth conditions than exposing the microplates to various amounts of airflow. The present invention implements shelves with exposure holes on all sides. Surprisingly, this has resulted in more uniform growth conditions for the microplates by allowing for airflow around and between all microplates. Thus, the inventive technical feature of the present invention contributed to a surprising result.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a schematic of an incubator rack of the present invention.

FIG. 1B shows a non-limiting embodiment of the incubator rack of the present invention.

FIG. 2 shows the incubator rack with microplates disposed therein.

FIG. 3 shows examples of incubator racks in the prior arts.

FIG. 4 shows a top-down view of the incubator rack with microplates disposed therein.

### DETAILED DESCRIPTION OF THE INVENTION

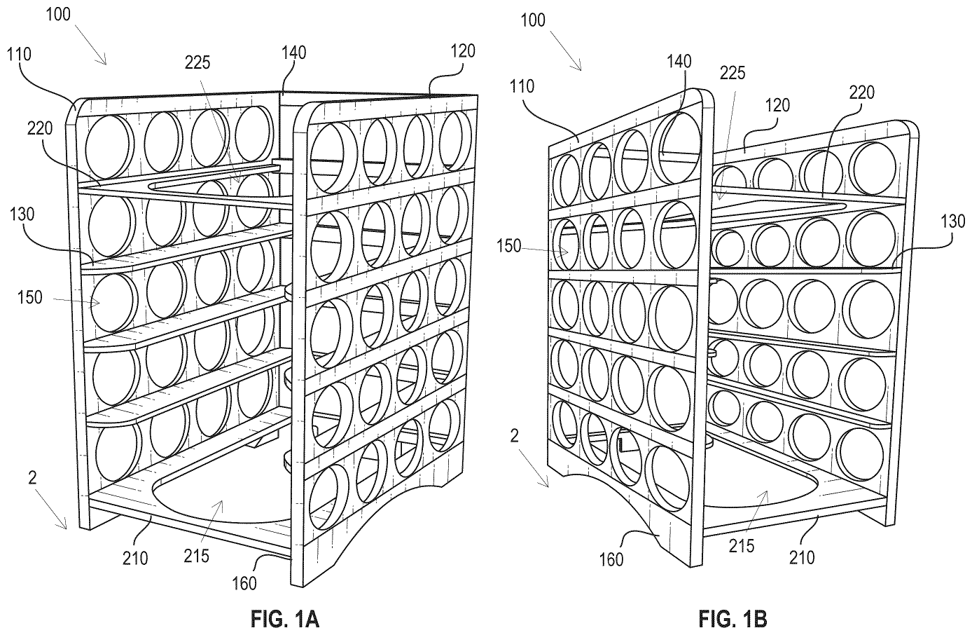

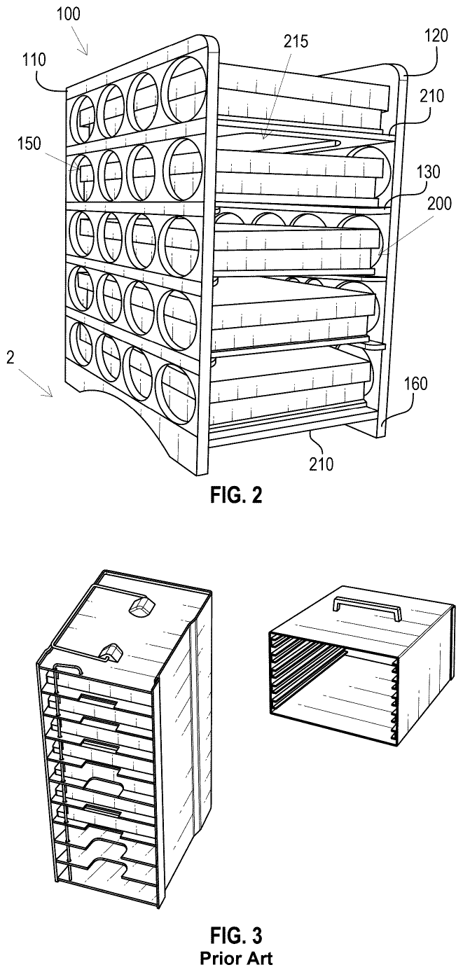

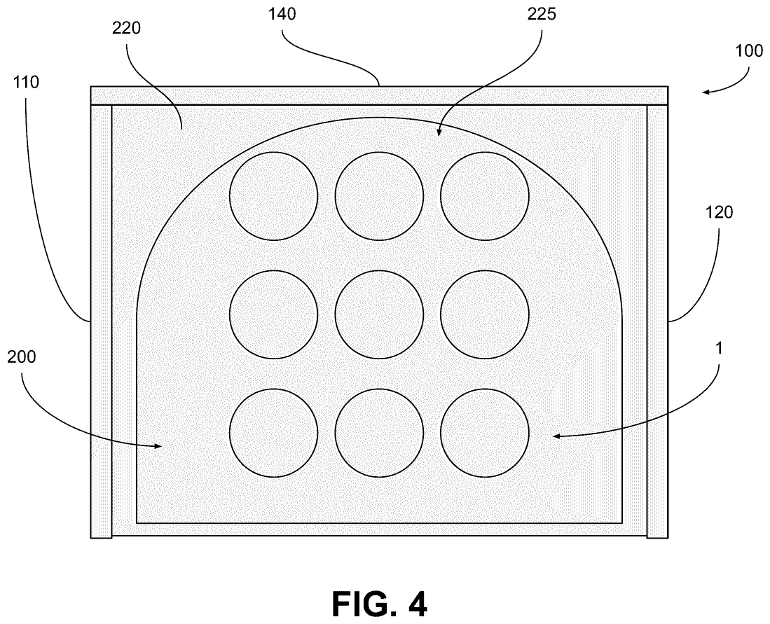

Following is a list of elements corresponding to a particular element referred to herein:
1 microplates
2 base surface
100 incubator rack
110 first sidewall
120 second sidewall
130 rails
140 sidewall frame
150 holes
160 feet
200 shelves
210 bottom shelf
215 through hole
220 top shelf
225 through hole
Referring now to FIGS. 1A-2, the present invention features an incubator rack (100) for providing uniform growth conditions for microplates (1) stored therein. The incubator rack (100) may comprise a plurality of shelves (200) for said microplates (1). The incubator rack (100) may comprise a plurality of holes (150) on all sides for airflow between and around the microplates (1). The incubator rack (100) may be placed on a base surface (2) such that a bottom shelf (210) of the incubator rack (100) is raised away from the base surface (2).

In some embodiments, the growth conditions may comprise airflow, temperature, growth rates, thermal rates, incubation time, and humidity. In some embodiments, the plurality of shelves (200) may be equally-spaced. In some embodiments, a height of the plurality of shelves (200) may range from 25 mm to 35 mm. In some embodiments, the bottom shelf (210) may be raised at least 10 mm away from the base surface (2). In some embodiments, the base surface (2) may be a surface of an incubator. In some embodiments, the incubator rack (100) may further comprise one or more feet (160) disposed under the bottom shelf (210) such that the one or more feet (160) raise the incubator rack (100) from the base surface (2). In some embodiments, the incubator rack (100) may be constructed from a material having low thermal conductivity.

The present invention features an incubator rack (100). The incubator rack (100) may comprise a first sidewall (110). The incubator rack (100) may further comprise a second sidewall (120) parallel to the first sidewall (110). The incubator rack (100) may further comprise a bottom shelf (210) connecting the first (110) and second (120) sidewalls. The bottom shelf (210) may have at least one through hole (215). The incubator rack (100) may be placed on a base surface (2) such that a bottom shelf (210) of the incubator rack (100) may be raised away from the base surface (2). The incubator rack (100) may further comprise a top shelf (220) connecting the first (110) and second (120) sidewalls. The top shelf (220) may be parallel to the bottom shelf (210), wherein the top shelf (220) has at least one through hole (225). The incubator rack (100) may further comprise a plurality of rails (130) disposed on an interior surface of each sidewall. Each rail may extend from one side edge to the other side edge of the sidewall. The rails may be disposed between the top and bottom shelves. Each rail on the first sidewall (110) may be aligned with a rail of the second sidewall (120) to form rail pairs. The bottom shelf (210), the top shelf (220), and the rail pairs may divide an interior space of the incubator rack (100) into one or more layers. The incubator rack (100) may further comprise a plurality of rows of holes (150) disposed through the first (110) and second (120) sidewalls such that at least one row of holes is disposed through the first sidewall (110) and the second sidewall (120) in each layer. The plurality of rows of holes (150) and the through holes may be effective for increasing airflow through the incubator rack (100) and lowering a thermal conductivity of the incubator rack (100).

In some embodiments, the through hole (215) of the bottom shelf (210) is U-shaped. In some embodiments, the through hole (215) of the bottom shelf (210) may occupy at least 50% of the bottom shelf (210). In some embodiments, the through hole (225) of the top shelf (220) may be U-shaped. In some embodiments, the through hole (225) of the top shelf (220) may occupy at least 50% of the top shelf (220). In some embodiments, a row of holes of each sidewall may be aligned with all other rows of holes of the sidewall. In some embodiments, a row of holes of each sidewall may be offset from adjacent rows of holes. In some embodiments, the plurality of holes in each sidewall may occupy at least 50% of the sidewall. In some embodiments, the one or more layers may be configured to receive microplates (1). In some embodiments, the incubator rack (100) may be constructed from a material having low thermal conductivity.

The present invention features an incubator rack (100). The incubator rack (100) may comprise a first sidewall (110). The incubator rack (100) may further comprise a second sidewall (120) parallel to the first sidewall (110). The incubator rack (100) may further comprise a bottom shelf (210) connecting the first (110) and second (120) sidewalls. The bottom shelf (210) may have at least one through hole (215) occupying at least 50% of a surface of the bottom shelf (210). The incubator rack (100) may be placed on a base surface (2) such that a bottom shelf (210) of the incubator rack (100) is raised away from the base surface (2). The incubator rack (100) may further comprise one or more feet (160) disposed under the bottom shelf (210) such that the one or more feet (160) raise the incubator rack (100) from the base surface (2). The incubator rack (100) may further comprise a top shelf (220) connecting the first (110) and second (120) sidewalls. The top shelf (220) may be parallel to the bottom shelf (210). The top shelf (220) may have at least one through hole (225) occupying at least 50% of a surface of the top shelf (220). The incubator rack (100) may further comprise a plurality of rails (130) disposed on an interior surface of each sidewall. Each rail may extend from one side edge to the other side edge of the sidewall. The rails (130) may be disposed between the top and bottom shelves. Each rail on the first sidewall (110) may be aligned with a rail of the second sidewall (120) to form rail pairs. The bottom shelf (210), the top shelf (220), and the rail pairs may divide an interior space of the incubator rack (100) into one or more layers.

The incubator rack (100) may further comprise a sidewall frame (140) perpendicular to the two parallel sidewalls, the top shelf (220), and the bottom shelf (210). The sidewall frame (140) may comprise a plurality of parallel bars, each spanning from the first sidewall (110) to the second sidewall (120). The incubator rack (100) may further comprise a plurality of rows of holes (150) disposed through the first (110) and second (120) sidewalls such that at least one row of holes is disposed through the first sidewall (110) and the second sidewall (120) in each layer. The plurality of rows of holes (150) and the through holes may be effective for increasing airflow through the incubator rack (100) and lowering a thermal conductivity of the incubator rack (100). The one or more layers may be configured to receive microplates (1). Each parallel bar may be aligned with a top edge of the sidewalls, a rail pair, or shelf.

In some embodiments, the present invention features an incubator rack for providing uniform growth conditions to microplates stored therein. The incubator rack may comprise a plurality of shelves for holding said microplates. Without wishing to limit the invention, the incubator rack can have adequate shelving space for sufficient airflow between and around the microplates. In other embodiments, the shelves have ventilation to provide said airflow.

In some embodiments, the incubator rack is constructed from a material having low thermal conductivity. In one embodiment, the material is a polymer. In some embodiments, the material may comprise polyamides, thermoplastic elastomers, polyaryletherketones, polylactic acid, polyethylene terephthalate glycol-modified polymers, acrylonitrile butadiene styrene, acrylonitrile styrene acrylate, polypropylene, polyamide, polycarbonate, polyetheretherketone, polyaryletherketones, polystyrene, or a combination thereof. In some embodiments, the incubator rack can be constructed by molding or 3D printing.

In some embodiments, a bottom shelf of the incubator rack is raised away from a base surface. The base surface may be a surface of an incubator. In some embodiments, the bottom shelf may be raised at least 5 mm or at least 10 mm away from the base surface. In some embodiments, the bottom shelf may be raised from the base surface by a distance of at least a third of the height of a layer. In some embodiments, the bottom shelf may be raised by one or more feet disposed on a bottom surface of the bottom shelf. In some embodiments, the rack may be suspended by the sidewalls, the top shelf, the sidewall frame, or a combination thereof such that the bottom shelf is raised from the base surface.

In other embodiments, there is a gap or space between an undersurface of a microplate and the lid of a microplate directly below the undersurface. In some other embodiments, the gap between two neighboring microplates in the incubator rack is at least about 3 mm.

In some embodiments, the incubator rack may comprise 1-15 shelves. In non-limiting embodiments, a height of each shelf can range from about 20 mm to 40 mm, or about 25 mm to 35 mm, or about 30 mm to 40 mm. In some embodiments, the shelves are equally-spaced. In other embodiments, the shelves have sufficient space to allow for a variety of lid types for the microplates.

In some embodiments, each shelf of the incubator rack may comprise a plurality of holes disposed on its sides. For example, each side of the shelf has a row of circular holes. However, the holes may take on any shape suitable to allow for airflow to the microplates. The number of holes may range from 3-10 holes per row. In some embodiments, holes may be of equal size. In some embodiments, the holes may vary in size. The size of holes may be dependent on the contents of the microplates.

The microplates may support cultures of mammalian, insect, plant, or microbial cells. In some embodiments, the microplates on the rack may all support the same cell culture type. In some embodiments, the microplates on the rack may all support various cell culture types.

Non-limiting examples of growth conditions include airflow, temperature, growth rates, thermal rates (i.e. heating and cooling rates), incubation time, and humidity. Without wishing to limit the invention, the incubation rack provides consistent growth conditions to the microplates, regardless of their placement in the incubator rack.

In some embodiments, the incubator rack further comprises a plurality of feet disposed under the bottom shelf. Preferably, the feet are sized and shaped so that they do not get stuck, caught, or trapped in holes, slits, ridges, or grooves disposed on the base surface. For example, at least one of a width or length of each foot is sized larger than an aperture of the base surface so that the foot does not get caught in the aperture. In non-limiting embodiments, the width of each foot ranges from about 5 mm to 20 mm. In other embodiments, the length of each foot ranges from about 5 mm to 20 mm.

In some embodiments, a height of the feet is sufficient to raise the bottom shelf away from the base surface. For example, the height of each foot is at least 5 mm or at least 10 mm.

According to other embodiments, the present invention features an incubator rack comprising a first sidewall, a second sidewall parallel to the first sidewall, a bottom shelf connecting the first and second sidewalls, a top shelf connecting the first and second sidewalls, a plurality of rails disposed on an interior surface of each sidewall, a sidewall frame perpendicular to the two parallel sidewalls, the top shelf, and the bottom shelf, and a plurality of holes disposed through the first and second sidewalls.

In some embodiments, the bottom shelf has at least one through hole occupying at least 50% of a surface of the bottom shelf. In other embodiments, the through hole of the bottom surface occupies at least 80% of the surface of the bottom shelf. In some embodiments, the through hole of the bottom shelf is U-shaped. In other embodiments, the through hole of the bottom shelf is circular or oblong shaped.

In some embodiments, the top shelf is parallel to the bottom shelf. In some embodiments, the top shelf has at least one through hole occupying at least 50% of a surface of the top shelf. In other embodiments, the through hole of the top surface occupies at least 80% of a surface of the top shelf. In other embodiments, the through hole of the top shelf is U-shaped. In some embodiments, the through hole of the top shelf is circular or oblong shaped.

In some embodiments, each rail extends from one side edge to the other side edge of the sidewall. The rails are disposed between the top and bottom shelves. In some embodiments, each rail on the first sidewall is aligned with a rail of the second sidewall to form rail pairs.

In some embodiments, the bottom shelf, the top shelf, and the rail pairs divide an interior space of the incubator rack into a plurality of layers. The layers are configured to receive microplates. The microplates may be supported by the rail pair or shelf.

In some embodiments, the sidewall frame comprises a plurality of parallel bars. Each parallel bar can span from the first sidewall to the second sidewall. Each parallel bar may be aligned with a top edge of the sidewalls, a rail pair, or shelf. The parallel bars act as a stopper to prevent the microplates from sliding out of the rack.

In some embodiments, at least one row of holes is disposed through the first sidewall and the second sidewall in each layer. Without wishing to limit the present invention, the plurality of holes and the through holes are effective for increasing airflow through the incubator rack and lowering a thermal conductivity of the incubator rack.

In some embodiments, a row of holes is aligned with the holes of the other rows. In other embodiments, a row of holes is offset from the holes of neighboring rows. In some embodiments, each row of holes comprises 3-6 holes. In some embodiments, the plurality of holes in each sidewall occupies at least 50% of the sidewall. In other embodiments, the plurality of holes in each sidewall occupies at least 80% of the sidewall.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An incubator rack (100) for providing uniform growth conditions for microplates (1) stored therein, said incubator rack (100) comprising a plurality of shelves (200) for said microplates (1), said incubator rack (100) comprising a plurality of holes (150) on all sides for airflow between and around the microplates (1), wherein the incubator rack (100) is placed on a base surface (2) such that a bottom shelf (210) of the incubator rack (100) is raised away from the base surface (2).

2. The incubator rack (100) of claim 1, wherein the growth conditions comprise airflow, temperature, growth rates, thermal rates, incubation time, and humidity.

3. The incubator rack (100) of claim 1, wherein the plurality of shelves (200) are equally-spaced.

4. The incubator rack (100) of claim 1, wherein a height of the plurality of shelves (200) ranges from 25 mm to 35 mm.

5. The incubator rack (100) of claim 1, wherein the bottom shelf (210) is raised at least 10 mm away from the base surface (2).

6. The incubator rack (100) of claim 1, wherein the base surface (2) is a surface of an incubator.

7. The incubator rack (100) of claim 1, further comprising one or more feet (160) disposed under the bottom shelf (210) such that the one or more feet (160) raise the incubator rack (100) from the base surface (2).

8. The incubator rack (100) of claim 1, wherein the incubator rack (100) is constructed from a material having low thermal conductivity.

9. An incubator rack (100) comprising:
a) a first sidewall (110);
b) a second sidewall (120) parallel to the first sidewall (110);
c) a bottom shelf (210) connecting the first (110) and second (120) sidewalls, wherein the bottom shelf (210) has at least one through hole (215), wherein the incubator rack (100) is placed on a base surface (2) such that a bottom shelf (210) of the incubator rack (100) is raised away from the base surface (2);
d) a top shelf (220) connecting the first (110) and second (120) sidewalls, wherein the top shelf (220) is parallel to the bottom shelf (210), wherein the top shelf (220) has at least one through hole (225);
e) a plurality of rails (130) disposed on an interior surface of each sidewall, wherein each rail extends from one side edge to the other side edge of the sidewall, wherein the rails are disposed between the top and bottom shelves, wherein each rail on the first sidewall (110) is aligned with a rail of the second sidewall (120) to form rail pairs,
wherein the bottom shelf (210), the top shelf (220), and the rail pairs divides an interior space of the incubator rack (100) into one or more layers;
f) a plurality of rows of holes (150) disposed through the first (110) and second (120) sidewalls such that at least one row of holes is disposed through the first sidewall (110) and the second sidewall (120) in each layer,
wherein the plurality of rows of holes (150) and the through holes are effective for increasing airflow through the incubator rack (100) and lowering a thermal conductivity of the incubator rack (100).

10. The incubator rack (100) of claim 9, wherein the through hole (215) of the bottom shelf (210) is U-shaped.

11. The incubator rack (100) of claim 9, wherein the through hole (215) of the bottom shelf (210) occupies at least 50% of the bottom shelf (210).

12. The incubator rack (100) of claim 9, wherein the through hole (225) of the top shelf (220) is U-shaped.

13. The incubator rack (100) of claim 9, wherein the through hole (225) of the top shelf (220) occupies at least 50% of the top shelf (220).

14. The incubator rack (100) of claim 9, wherein a row of holes of each sidewall is aligned with all other rows of holes of the sidewall.

15. The incubator rack (100) of claim 9, wherein a row of holes of each sidewall is offset from adjacent rows of holes.

16. The incubator rack (100) of claim 9, wherein the plurality of holes in each sidewall occupies at least 50% of the sidewall.

17. The incubator rack (100) of claim 9, wherein the one or more layers are configured to receive microplates (1).

18. The incubator rack (100) of claim 9, wherein the incubator rack (100) is constructed from a material having low thermal conductivity.

19. An incubator rack (100) comprising:
a) a first sidewall (110);
b) a second sidewall (120) parallel to the first sidewall (110);
c) a bottom shelf (210) connecting the first (110) and second (120) sidewalls, wherein the bottom shelf (210) has at least one through hole (215) occupying at least 50% of a surface of the bottom shelf (210), wherein the incubator rack (100) is placed on a base surface (2) such that a bottom shelf (210) of the incubator rack (100) is raised away from the base surface (2);
d) one or more feet (160) disposed under the bottom shelf (210) such that the one or more feet (160) raise the incubator rack (100) from the base surface (2);
e) a top shelf (220) connecting the first (110) and second (120) sidewalls, wherein the top shelf (220) is parallel to the bottom shelf (210), wherein the top shelf (220) has at least one through hole (225) occupying at least 50% of a surface of the top shelf (220);
f) a plurality of rails (130) disposed on an interior surface of each sidewall, wherein each rail extends from one side edge to the other side edge of the sidewall, wherein the rails (130) are disposed between the top and bottom shelves, wherein each rail on the first sidewall (110) is aligned with a rail of the second sidewall (120) to form rail pairs,
wherein the bottom shelf (210), the top shelf (220), and the rail pairs divides an interior space of the incubator rack (100) into one or more layers;
g) a sidewall frame (140) perpendicular to the two parallel sidewalls, the top shelf (220), and the bottom shelf (210), wherein the sidewall frame (140) comprises a plurality of parallel bars, each spanning from the first sidewall (110) to the second sidewall (120); and
h) a plurality of rows of holes (150) disposed through the first (110) and second (120) sidewalls such that at least one row of holes is disposed through the first sidewall (110) and the second sidewall (120) in each layer,
wherein the plurality of rows of holes (150) and the through holes are effective for increasing airflow through the incubator rack (100) and lowering a thermal conductivity of the incubator rack (100),
wherein the one or more layers are configured to receive microplates (1).

20. The incubator rack (100) of claim 19, wherein each parallel bar is aligned with a top edge of the sidewalls, a rail pair, or shelf.
